SEQUENCE LISTING (6 sequences)

SEQ ID NO: 1 - protein, 22 aa, synthetic construct
AVVQDPALKPLALVYGEATSRR

SEQ ID NO: 2 - protein, 9 aa, synthetic construct
VLDALDSIK

SEQ ID NO: 3 - protein, 15 aa, synthetic construct
DYWTYPGSLTTPPLL

SEQ ID NO: 4 - protein, 20 aa, synthetic construct
DYWTYPGSLTTPPLLESVTW

SEQ ID NO: 5 - protein, 17 aa, synthetic construct
DYWTYPGSLTTPPLLES

SEQ ID NO: 6 - protein, 260 aa, Bos taurus
MSHHWGYGKHNGPEHWHKDFPIANGERQSPVDIDTKAVVQDPALKPLALVYGEATSRRMVNNGHSFNVEYDDSQDKAVLKDGPLTGTYRLVQFHFHWGSSDDQGSEHTVDRKKYAAELHLVHWNTKYGDFGTAAQQPDGLAVVGVFLKVGDANPALQKVLDALDSIKTKGKSTDFPNFDPGSLLPNVLDYWTYPGSLTTPPLLESVTWIVLKEPISVSSQQMLKFRTLNFNAEGEPELLMLANWRPAQPLKNRQVRGFPK

(19) United States
(12) Patent Application Publication
(10) Pub. No.: US 2026/0219279 A1
(43) Pub. Date: Jul. 30, 2026

(54) IMPROVED HIGH-THROUGHPUT HYDROGEN-DEUTERIUM EXCHANGE-MASS SPECTROMETRY SYSTEM AND METHOD

(71) Applicant: THE BOARD OF REGENTS OF THE UNIVERSITY OF OKLAHOMA, Norman, OK (US)

(72) Inventors: Si WU, Norman, OK (US); Mulin FANG, Norman, OK (US); Oliver Y. WU, Norman, OK (US); Kellye A. CUPP-SUTTON, Newcastle, OK (US)

(73) Assignee: THE BOARD OF REGENTS OF THE UNIVERSITY OF OKLAHOMA, Norman, OK (US)

(21) Appl. No.: 19/143,473

(22) PCT Filed: Dec. 21, 2023
(86) PCT No.: PCT/US2023/085371

§ 371 Date: Jun. 26, 2025

Related U.S. Application Data
(60) Provisional application No. 63/436,651, filed on Jan. 2, 2023.

(51) Int. Cl. G01N 33/68; G01N 1/40; G01N 30/06; G01N 30/72

(52) U.S. Cl. CPC G01N 33/6848; G01N 1/4022; G01N 1/4044; G01N 30/06; G01N 30/7233; G01N 2030/067; G01N 2458/15

(57) ABSTRACT

An improved system and method for analyzing highly complex biological samples such as cell lysates and other mixtures which contain proteins. The disclosed system and method incorporates a thermal protein depletion step prior to subzero temperature long gradient UPLC-HDX-MS (ultra-performance liquid chromatography hydrogen deuterium exchange coupled with mass spectrometry) to substantially reduce protein content and complexity of the mixture so that analysis of particular proteins can be enhanced. In particular, the system and method is used to facilitate high-throughput analysis of protein-ligand interactions, for example for the identification of ligand targets and characterization of protein-ligand interactions such as identification of binding sites in cell lysates and other complex biological mixtures.

### CROSS REFERENCE TO RELATED APPLICATIONS/INCORPORATION BY REFERENCE STATEMENT

The present patent application claims priority to United States Provisional Patent Application U.S. Ser. No. 63/436,651, filed on Jan. 2, 2023, the entire contents of which is hereby expressly incorporated herein by reference.

### STATEMENT REGARDING FEDERALLY SPONSORED RESEARCH OR DEVELOPMENT

This invention was made with government support under grant numbers R01AI141625 and 2U19AI062629 awarded by the NIAID of the National Institutes of Health. The government has certain rights in the invention.

### REFERENCE TO A SEQUENCE LISTING

This application contains a Sequence Listing XML, which has been submitted electronically and is hereby incorporated by reference in its entirety. Said Sequence Listing XML, created on Dec. 20, 2023, is named OKLAP0014WO_ST26.xml and is 7 KB in size.

### BACKGROUND

Hydrogen-deuterium exchange coupled with mass spectrometry (HDX-MS) is a powerful protein footprinting approach for the characterization of protein-ligand interactions¹. This technique is based on the dependence of the exchange rate of protein amide hydrogens with deuterium on solvent accessibility and hydrogen bonding when the protein is exposed to deuterium oxide²⁻³. Consequently, reduced solvent accessibility following ligand binding protects the amide hydrogens involved in the binding interface against exchange while the same amide hydrogens will undergo rapid exchange without ligand binding. In the typical HDX-MS workflow, deuterium labeling is initiated by the dilution of the protein into a deuterium oxide-prepared buffer, so that proteins maintain native state. The deuterium labeling reaction is then quenched by decreasing the pH to 2.5. After quenching, proteins are digested using an acid-stable protease and subjected to LC separation and MS detection⁴⁻⁵. Over the recent decades, improvements in several aspects of HDX sample handling including protein reduction and digestion⁶⁻⁹, separation¹⁰⁻¹², MS detection¹³, and HDX data analysis software¹⁴⁻¹⁶ have enabled the HDX-MS analysis of a wide size range of proteins under native conditions where other traditional methods for the characterization of protein-ligand interaction may be of limited use¹⁷. Currently, the HDX-MS community seeks to investigate protein-ligand interactions in native-like biological environments such as cell lysates^{16, 18-19}.

Back exchange, or the loss of deuterium after quenching, is a particularly confounding issue in conventional HDX-MS analysis²⁰. Back exchange can reduce sensitivity and make it difficult to distinguish HDX variation between protein and protein-ligand complexes which can bias the identification of sites of interest. Thus, a short LC gradient is typically implemented (e.g., 5-10 minutes) to reduce spectral complexity and minimize back exchange¹⁷. However, short LC gradients can hinder the application of HDX-MS analysis to highly complex protein matrix environments, such as cell lysates, as the deuterium labeled peptides in a mixture of all digested proteins will coelute together in a short LC gradient, resulting in extreme spectral complexity that compromises the HDX data analysis^{13, 19, 21}. Furthermore, as with data dependent acquisition-based shotgun proteomics, sequence coverage of the protein(s) of interest will be decreased in the presence of a large amount of co-eluted peptides from nontarget proteins²²⁻²³. Immobilization²⁴⁻²⁵ or affinity capture²² of nontargeted proteins and the integration of size exclusion chromatography (SEC)²¹ prior to LC-MS analysis have been developed for HDX-MS analysis to address high spectral complexity. However, reported strategies for protein enrichment, including immobilization and biotinylation-based affinity capture of proteins, may introduce steric effects resulting in changes to protein-ligand interactions. Short SEC separations may suffer from low-resolution for highly complex biological samples such as cell lysates. Moreover, these strategies are typically only applicable to purified protein systems with known target proteins and are unsuitable for HDX-MS analysis of unknown protein-ligand interactions in complex samples such as cell lysates.

To improve separation power, ion mobility²⁶, UPLC-based separation¹⁰⁻¹¹, and subzero temperature-based long gradient separations^{18-19, 27} have been introduced into the HDX-MS workflow. Previously, our group optimized a long gradient (e.g., 90 minutes) subzero-temperature UPLC separation method using E. coli cell lysate digest, which significantly boosts LC separation power to enable the high-throughput analysis of thousands of peptides with low back exchange in a single HDX-MS analysis for complex biological samples¹⁹. Yet, despite progress, HDX-MS analysis is still not well-adapted for the characterization of protein-ligand interactions in native-like biological environments such as cell lysates or complex protein mixtures. Therefore, improved systems and methods for HDX-MS characterization of protein-ligand interactions in native-like biological environments would be highly useful. It is to such improved systems and methods that the present disclosure is directed.

### BRIEF DESCRIPTION OF THE DRAWINGS

Several embodiments of the present disclosure are hereby illustrated in the appended drawings. It is to be noted however, that the appended drawings only illustrate several typical embodiments and are therefore not intended to be considered limiting of the scope of the inventive concepts disclosed herein. The figures are not necessarily to scale and certain features and certain views of the figures may be shown as exaggerated in scale or in schematic in the interest of clarity and conciseness.

FIG. 1 shows results of an evaluation of the melting points of Call in an E. coli cell lysate not treated with AZM.

FIG. 2 shows results of an evaluation of the melting points of Call in an E. coli cell lysate treated with AZM.

FIG. 3 shows validation of AZM target using two-temperature-points thermal proteome profiling. (A) Volcano plot for comparing the intensities between the identified E. coli peptides with and without AZM treated at 37° C. The dashed lines indicate the cut off values (fold change>2, P-value<0.01). (B) Volcano plot for comparing the intensities between the identified E. coli peptides with and without AZM treatment after protein thermal depletion at 70° C. The dashed lines indicate the cut off values (fold change>2, P-value<0.01). Arrows point to two Call peptides (AVVQDPALKPLALVYGEATSRR (SEQ ID NO: 1) and VLDALDSIK (SEQ ID NO: 2)). (C) EICs of peptide AVVQDPALKPLALVYGEATSRR (SEQ ID NO: 1) at 37° C. and 70° C. (experiments were in triplicate).

FIG. 4 shows an SDS-PAGE evaluation of heated E. coli cell lysates not treated with AZM.

FIG. 5 shows an SDS-PAGE evaluation of heated E. coli cell lysates treated with AZM.

FIG. 6 shows BCA evaluation of protein concentration of E. coli cell lysates (not treated with AZM) before PTD (37° C.) and after PTD at 60° C.

FIG. 7 shows a comparison of the number of identified Call peptides in E. coli cell lysate before PTD (37° C.) and after PTD at 60° C. following the HDX-MS workflow.

FIG. 8A shows a map of Call peptides identified in E. coli cell lysates heated at 37° C. The bars represent the Call peptides identified in both E. coli cell lysates. The protein sequence is SEQ ID NO: 6.

FIG. 8B shows a map of Call peptides identified in E. coli cell lysates heated at 60° C. (B). The bars represent the Call peptides identified in both E. coli cell lysates. The protein sequence is SEQ ID NO: 6.

FIG. 9A shows LC-HDX-MS analysis of Call spiked E. coli cell lysate digest with different gradient lengths. Base peak chromatograms under different gradient lengths are shown.

FIG. 9B shows peptide counts from E. coli cell lysates of FIG. 9A.

FIG. 9C shows identified Call peptides from E. coli cell lysates of FIG. 9A.

FIG. 10 shows PTD-HDX-MS for the elucidation of the AZM target and mechanism-of-action in 60° C.-depleted cell lysate. (A) Volcano plot showing deuterium uptake differences of E. coli peptides with/without AZM treatment under different HDX labeling conditions (0.3 Da deuterium uptake difference and p value<0.01 are set as cutoffs). Six Call peptides exhibited significantly reduced deuterium uptake after AZM treatment (B) Structural interpretation of HDX-MS analysis of Call (PDB:1V9E). The cyan color indicates no significant deuterium uptake difference in this region. The red color indicates significant deuterium uptake differences for peptides characterized in this region. The grey color indicates no coverage in the region.

FIG. 11 shows MS spectra of the peptide Call (189-203) DYWTYPGSLTTPPLL (SEQ ID NO: 3) in triplicate under different HDX labeling conditions.

FIG. 12A shows MS spectra of the peptides Call (189-208) DYWTYPGSLTTPPLLESVTW (SEQ ID NO: 4) and Call (189-205) DYWTYPGSLTTPPLLES (SEQ ID NO: 5) in triplicate under 90 s HDX labeling.

FIG. 12B shows MS spectra of the peptides Call (189-208) DYWTYPGSLTTPPLLESVTW (SEQ ID NO: 4) and Call (189-205) DYWTYPGSLTTPPLLES (SEQ ID NO: 5) in triplicate under 120 s HDX labeling.

FIG. 13 shows examples of structural interpretation of PTD-HDX-MS analysis of other E. coli proteins. (A) Alkyl hydroperoxide reductase C (AHPC). (B) 60 kDa chaperonin (CH60). (C) UPF0337 protein YjbJ (YJBJ). (D) Glyceraldehyde-3-phosphate dehydrogenase A (G3P1). The cyan color indicates no significant deuterium uptake difference in this region. The grey color indicates no coverage in the region.

FIG. 14 shows a Woods' plot from the HDX-MS analysis of pure Call bound with AZM. The dashed and full lines indicate 98% and 99% confidence, respectively. Peptides with significant shifts in deuteration are designated in red and located in the lower righthand corner of the plot (with less than −0.4 Da difference), whereas the other bars above −0.2 Da difference represent peptides without significant differential deuterium uptake.

FIG. 15 shows MS spectra of Call involved in binding site in triplicate under 120 s HDX labeling conditions in pure protein complex. Call (189-203): DYWTYPGSLTTPPLL (SEQ ID NO: 3); Call (189-205): DYWTYPGSLTTPPLLES (SEQ ID NO: 5); Call (189-208): DYWTYPGSLTTPPLLESVTW (SEQ ID NO: 4).

FIG. 16A shows results of an evaluation of conformations of Call under two conditions: Call at 37° C., and Call cooled down after heating at 60° C. for 5 minutes. (A) Overall deuterium uptake of Call peptides.

FIG. 16B shows results of the analysis of FIG. 17A. Statistical analysis of deuterium uptake differences of Call peptides using results of the analysis of FIG. 17A. (0.3 Da deuterium uptake difference and p-value<0.01 are set as cutoffs).

FIG. 16C shows a comparison of isothermal titration calorimetry of Call upon on AZM binding at 37° C. and 60° C.

FIG. 16D shows a comparison of isotherm profiles of Call binding with AZM at 37° C. and 60° C.

The following abbreviations may be used herein:
ABC: ammonia bicarbonate,
ABPP: activity-based protein profiling,
AGC: automatic gain control,
AZM: Acetazolamide,
BCA: Bicinchoninic acid,
Call: carbonic anhydrase II,
CETSA: cellular thermal shift assay,
cDNA: complementary deoxyribonucleic acid,
DIA: Data-independent acquisition,
D₂O: deuterium oxide,
DNA: deoxyribonucleic acid,
DTT: dithiothreitol,
E. coli: Escherichia coli
EIC: Extracted ion chromatograph,
FDR: false discovery rate,
HCD: higher-energy C-trap dissociation,
HDX: Hydrogen-deuterium exchange,
HDX-MS: Hydrogen-deuterium exchange coupled with mass spectrometry,
HPLC: high performance liquid chromatography,
IEC: ion exchange chromatography,
ITC: isothermal titration calorimetry,
LC: liquid chromatography,
LFQ: label-free quantitation,
mRNA: messenger ribonucleic acid,
MS: mass spectrometry,
NMR: nuclear magnetic resonance,
PBS: phosphate buffered saline,
PMSF: phenylmethylsulfonyl fluoride,
PTD: protein thermal depletion,
RLPC: reverse phase liquid chromatography,
RNA: ribonucleic acid,
SDS-PAGE: sodium dodecyl sulfate polyacrylamide gel electrophoresis,
SEC: size exclusion chromatography,
shRNA: short hairpin ribonucleic acid,
siRNA: small interfering ribonucleic acid,
TCEP: Tris(2-carboxyethyl)phosphine,
Tₘ: protein melting (denaturation) point,
TPCA: thermal proximity coaggregation,
TPP: thermal proteome profiling,
UPLC: ultra-performance liquid chromatography.

### DETAILED DESCRIPTION

The present disclosure is directed to an improved system and method for analyzing highly complex biological samples such as cell lysates and other mixtures which contain proteins. The disclosed system and method incorporate a protein thermal depletion (PTD) process with subzero temperature long gradient UPLC-HDX-MS (ultra-performance liquid chromatography hydrogen-deuterium exchange coupled with mass spectrometry) to substantially reduce protein content and complexity of the mixture so that analysis of particular proteins can be improved. In particular, the system and method is used to facilitate high-throughput analysis of protein-ligand interactions, for example for the identification of ligand targets and characterization of protein-ligand interactions such as identification of binding sites in cell lysates and other complex biological mixtures.

Before describing various embodiments of the present disclosure in more detail by way of exemplary description, examples, and results, it is to be understood that the present disclosure is not limited in application to the details of methods and compositions as set forth in the following description. The present disclosure is capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-25 therefore includes 1, 2, 3, 4, 5, 6, 7, 8, 9, 10, 11, 12, 13, 14, 15, 16, 17, 18, 19, 20, 21, 22, 23, 24, and 25, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100 units to 1000 units, 100 units to 500 units, 200 units to 1000 units, 300 units to 1500 units, 400 units to 2000 units, 500 units to 2000 units, 500 units to 1000 units, 250 units to 1750 units, 250 units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

A reference to fractions of liters, such as 1 pL to 100 μL is intended to explicitly include all quantities in the range, such as 10 pL to 1 μL. Where used herein, the term ultra low-volume sample refers to a sample having a volume in a range of 15 pL to 25 nL.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” and “approximately” are used to indicate that a value includes the inherent variation of error. Further, in this detailed description, each numerical value (e.g., temperature or time) should be read once as modified by the term “about” (unless already expressly so modified), and then read again as not so modified unless otherwise indicated in context. As noted, any range listed or described herein is intended to include, implicitly or explicitly, any number within the range, particularly all integers, including the end points, and is to be considered as having been so stated. For example, “a range from 1 to 10” is to be read as indicating each possible number, particularly integers, along the continuum between about 1 and about 10. Thus, even if specific data points within the range, or even no data points within the range, are explicitly identified or specifically referred to, it is to be understood that any data points within the range are to be considered to have been specified, and that the inventors possessed knowledge of the entire range and the points within the range. The use of the term “about” may mean a range including ±1%, ±5%, ±10%, ±15%, or ±20% of the subsequent number unless otherwise stated.

As used herein, the term “substantially” means that the subsequently described parameter, event, or circumstance completely occurs or that the subsequently described parameter, event, or circumstance occurs to a great extent or degree. For example, the term “substantially” means that the subsequently described parameter, event, or circumstance occurs at least 75% of the time, or at least 80% of the time, or at least 85% of the time, or at least 90% of the time, or at least 91%, or at least 92%, or at least 93%, or at least 94%, or at least 95%, or at least 96%, or at least 97%, or at least 98%, or at least 99%, of the time, or means that the dimension or measurement is within at least 75%, or at least 80%, or at least 85%, or at least 90%, or at least 91%, or at least 92%, or at least 93%, or at least 94%, or at least 95%, or at least 96%, or at least 97%, or at least 98%, or at least 99%, of the referenced dimension or measurement (e.g., length).

As used herein, the term “ligand” refers to a compound or biological molecule that binds with high affinity to another, usually larger, biomolecule, such as a protein or protein fragment. Ligands of the present disclosure include, for example, small molecules such as therapeutic drugs; dyes, stains, contrast agents, fluorescent compounds or molecules, or other diagnostic compounds; molecules constructed of amino acids, such as peptides, oligopeptides, polypeptides, proteins, antibodies, or antibody fragments; and nucleic acids such as DNA or RNA.

As used herein, the term “antibody” includes, but is not limited to, synthetic antibodies, monoclonal antibodies, recombinantly produced antibodies, intrabodies, multispecific antibodies (including bi-specific antibodies), human antibodies, humanized antibodies, chimeric antibodies, recombinant single chain polypeptide molecules in which light and heavy chain variable regions are connected by a peptide linker, i.e., single-chain Fv (scFv) fragments, bivalent scFv (bi-scFv), trivalent scFv (tri-scFv), Fab fragments, Fab′ fragments, F(ab′) fragments, F(ab′)₂ fragments, F(ab)₂ fragments, disulfide-linked Fvs (sdFv) (including bi-specific sdFvs), and anti-idiotypic (anti-Id) antibodies, dAb fragments, nanobodies, diabodies, triabodies, tetrabodies, linear antibodies, isolated CDRs, and epitope-binding fragments of any of the above. Regardless of structure, an antibody fragment refers to an isolated portion of the antibody that binds to the same antigen that is recognized by the intact antibody. Antibody fragments can be produced by recombinant DNA techniques or by enzymatic or chemical separation of intact immunoglobulins. Examples of antibody fragments include but are not limited to Fv, Fab, Fab′, Fab′-SH, F(ab′)2; diabodies; linear antibodies; single-chain antibody molecules (e.g., scFv); and multispecific antibodies formed from antibody fragments. The term “Fc region” herein is used to define a C-terminal region of an immunoglobulin heavy chain that contains at least a portion of the constant region. The term includes native sequence Fc regions and variant Fc regions.

The term “nucleic acid” as used herein refer to a molecule (i.e., a strand) of DNA, RNA or a derivative or analog thereof, comprising a nucleobase. A nucleobase includes, for example, a naturally-occurring purine or pyrimidine base found in DNA (e.g., an adenine “A,” a guanine “G,” a thymine “T” or a cytosine “C”) or RNA (e.g., an “A,” a “G,” a uracil “U” or a “C”). The term nucleobase also includes non-natural bases. The term “nucleic acid” encompasses the terms “oligonucleotide” and “polynucleotide,” each as a subgenus of the term “nucleic acid.” The term “oligonucleotide” generally refers to a molecule of between about 3 and about 100 nucleobases in length. The term “polynucleotide” generally refers to at least one molecule of greater than about 100 nucleobases in length. These definitions generally refer to a single-stranded molecule, but in specific embodiments will also encompass an additional strand that is partially, substantially or fully complementary to the single-stranded molecule. Thus, a nucleic acid may encompass a double-stranded molecule that comprises a complementary strand or “complement” of a particular sequence comprising a molecule. The terms “polynucleotide sequence” or “nucleic acid,” as used herein, include any polynucleotide sequence which encodes a peptide or fusion protein (or polypeptide) including polynucleotides in the form of RNA, such as mRNA, or in the form of DNA, including, for instance, cDNA and genomic DNA obtained by cloning or produced by chemical synthetic techniques or by a combination thereof. The RNA or DNA may be double-stranded or single-stranded. Single-stranded DNA may be the coding strand, also known as the sense strand, or it may be the non-coding strand, also referred to as the anti-sense strand. Oligonucleotides include for example, siRNA, shRNA, DNA, or RNA antisense oligonucleotide, chimeric antisense DNA/RNA, or microRNA, or other biochemical or molecule that is biologically active as described herein. By “biologically active” is meant the ability to modify the molecular, biochemical, or physiological system of a cell, organ, or organism, without reference to how the active agent has its physiological effects.

The term “antigen” as used herein, refers to any substance, including toxins and chemicals, that induces an immune response against that substance.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein any reference to “we” as a pronoun may include laboratory personnel or other contributors who assisted in the laboratory procedures and data collection and is not intended to represent an inventorship role by said laboratory personnel or other contributors in any subject matter disclosed herein.

Returning now to the description of particular embodiments of the present disclosure, it is known that shifts in protein thermal stability upon ligand binding have been widely used to study protein-ligand interactions²⁸⁻²⁹. The temperature at which a protein denatures (melting point, Tₘ) depends on the thermal stability of the protein. Upon exposure to heat at the melting point (Tₘ) or higher temperatures, proteins will denature and precipitate; precipitated proteins can be removed from the sample via filtration or centrifugation. However, it is possible that target proteins in the cell lysate will become more stable upon ligand binding and resist thermal denaturation at temperatures which more unstable proteins aggregate. It is reported that protein Tₘ is widely distributed between 45° C. to 80° C. for the E. coli proteome and 40° C. to 65° C. for the human (Homo sapiens) proteome.³⁰ Therefore, PTD by heating ligand-treated cell lysate at temperatures lower than the target protein Tₘ allows the removal of nontargeted proteins with lower Tₘ, providing a potential strategy to reduce sample complexity for high-throughput HDX-MS analysis in cell lysates. Here, we aim to integrate PTD with our subzero-temperature UPLC-HDX-MS platform (PTD-HDX-MS) for the high-throughput analysis of protein-ligand interactions in cell lysate.

### EXAMPLES

Novel embodiments of the present disclosure, having now been generally described, will be more readily understood by reference to the following examples and embodiments, which are included merely for purposes of illustration of certain aspects and embodiments of the present disclosure, and are not intended to be limiting. The following detailed examples and embodiments are to be construed, as noted above, only as illustrative, and not as limitations of the present disclosure in any way whatsoever. Those skilled in the art will promptly recognize appropriate variations from the various compositions, structures, components, procedures, and methods.

### EXPERIMENTAL

### Materials and Methods

Unless otherwise stated, all chemicals and enzymes including Call and AZM were acquired from Sigma-Aldrich (Milwaukee, WI). The ACE® Excel® SuperC18-AR™ column for UPLC separation was purchased from Advanced Chromatography Technologies Ltd (Aberdeen, Scotland). The Strata C18-U desalting column was acquired from Phenomenex (Torrance, CA). TCEP was purchased from Gold Biotechnology (St. Louis, MO).

E. coli Cell Lysate Preparation

Five μL of E. coli origin media was added to a pre-prepared lysogeny broth and shaken overnight at 250 rpm and 37° C. This solution was transferred to pre-prepared LB flasks and shaken overnight at 250 rpm and 37° C. The second inoculation solution was shaken for 15 minutes at 7800 rpm and 4° C. After supernatant removal, the resulting pellet was resuspended in 25 mM ABC buffer, and PMSF was added to achieve a final concentration of 0.1% (v/v). An Avestin C3 Emulsiflex homogenizer and 25 mM ABC buffer were used to lyse the E. coli cells. 1.5 mL aliquots were prepared in 1.5 mL Eppendorf tubes and centrifuged for one hour at 13,000 rpm and 4° C. The supernatant was transferred to fresh Eppendorf tubes for the following uses.

### Protein Thermal Depletion

A stock solution of Call was prepared to a concentration of 1 μg/μL in 1×PBS. Stock AZM solution was also prepared in 1×PBS to a concentration of 1 mM. 1.5 mL of solution containing AZM, Call, and E. coli cell lysate in PBS (+AZM) was prepared to final concentrations of 0.05 mM, 0.1 μg/μL, and about 2 μg/μL respectively. A second solution without AZM (−AZM) was prepared in PBS with the same concentrations of cell lysate proteins and Call. 100 μL aliquots of each solution were prepared into 0.2 mL PCR tubes. AZM+ and AZM− samples were heated in triplicate at an increasing temperature gradient from 37° C., 45-90° C. in 5° C. increments for 5 minutes using a PTC-200 Thermal Cycler. Samples were then allowed to return to room temperature for 3 minutes before being cooled on ice to 4° C. All tubes were centrifuged at 10,000 rpm for 30 minutes at 4° C. Without disturbing the aggregated peptides, 90 μL of the remaining supernatant were transferred to 0.5 mL Eppendorf tubes and stored at −80° C. before further analysis. Protein concentration of the depleted samples was measured using a Pierce™ BCA Protein Assay Kit and its provided protocol from Thermo Fisher Scientific.

### SDS-PAGE

SDS-PAGE was utilized to evaluate depleted E. coli samples. All chemicals and equipment related to SDS-PAGE were purchased from Bio-Rad technologies. 4× reducing buffer was prepared from 900 μL of 4× Laemmli buffer and 100 μL of 2-mercaptoethanol. 10 μL of each sample was added to 5 μL of reducing buffer and heated at 95° C. for 5 minutes. The denatured samples were injected into a 12% running and 4% stacking gel and subjected to 3.00 A of current at 120 V for 90 minutes using a PowerPac™ HC and Mini-PROTEAN Tetra Cell. Gels were stained using Coomassie Brilliant Blue R dye (Sigma-Aldrich, Milwaukee, WI).

### Subzero Temperature Liquid Chromatography HDX-MS

### Sample Preparation for the Assessment of PTD

The free Call sample (−AZM) at 37° C. (without PTD) and 60° C.-depleted (PTD at 60° C.) −AZM sample were used to assess PTD for HDX-MS analysis. Based on BCA results, 10 μL of each sample containing 13.5 g of E. coli proteins for each sample were prepared and then was diluted with 90 μL of 1×PBS in H₂O (pH 7.5) with the incubation time of 120 s at room temperature to mimic the HDX labeling process, followed by the addition of 100 μL of prechilled quenching solution containing 4M Urea and 200 mM TCEP in 1% formic acid (pH 2.5). The resulting solutions were incubated at 0° C. for 3 min. Next, protein digestion was performed by mixing 100 μL of 2.4 mg/mL protease type XIII with the quenched solution at 0° C. for 4 min. The digested solutions were immediately mixed with 30 μL of acetonitrile in 1% formic acid prior to subzero-temperature LC separation to avoid sample freezing. Triplicate experiments were performed for both 37° C. and 60° C.-depleted −AZM samples.

### Sample Preparation for PTD-HDX-MS Analysis in 60° C.-Depleted Samples

60° C.-depleted −AZM and 60° C.-depleted +AZM samples were used to perform differential PTD-HDX-MS. 10 μL of each sample was diluted with 90 μL of 1×PBS in D₂O (pH 7.5) and incubated at room temperature for various times (90 s, and 120 s). 100 μL of prechilled quenching solution containing 4M Urea and 200 mM TCEP in 1% formic acid (pH 2.5) was added to each sample and the total solution was incubated at 0° C. for 3 min. Subsequent protein digestion was performed by mixing 100 μL of 2.4 mg/mL protease type XIII with the quenched solution at 0° C. for 4 min, and 30 μL of acetonitrile in 1% FA was immediately mixed with the solution prior to subzero-temperature LC separation. Non-deuterated conditions were created for −AZM samples by using H₂O rather than D₂O to generate a library of E. coli peptides. Samples were prepared in triplicate.

### Sample Preparation for HDX-MS Analysis in Pure Cal and AZM

Pure Call solution was prepared to a concentration of 0.1 μg/μL in 1×PBS buffer. A CaII-AZM complex solution was prepared by incubating 0.1 μg/μL Call with 0.05 mM AZM in 1×PBS buffer. 10 μL of each solution was diluted with 90 μL of 1×PBS in D₂O (pH 7.5) and incubated at room temperature for 120 s. Quenching and digestion followed the exact steps in PTD-HDX-MS sample preparation. Non-deuterated conditions were created for pure Call by using H₂O rather than D₂O to generate a library of Call peptides. Samples were prepared in triplicate.

### Subzero Temperature LC Separation

Subzero temperature LC separation at −10° C. was achieved using a C18-AR UPLC column (100 mm×2.1 mm, 1.7 mm, 90 Å) placed in a portable freezer¹⁹. A Thermo Accela pump was used to control the gradient. Mobile phase A (0.1% formic acid and 10% acetonitrile in HPLC-grade water) and mobile phase B (0.1% formic acid in acetonitrile) were used for LC separation. The flowrate was controlled at 150 μL/min. Samples were loaded over 10 minutes with 0% mobile phase B. Peptides were then separated by an increase from 0% to 33% mobile phase B with a 15 min gradient for the PTD assessment or 45 min gradient for HDX-MS analysis. Then, the UPLC column was washed with 90% mobile phase B for 5 min followed by a decrease to 0% mobile phase B for 3 min and re-equilibration for 10 min. The eluted peptides were detected using an Orbitrap Exploris 240 mass spectrometer (Thermo Fisher Scientific, Bremen, Germany). Blank runs with the same gradient setup were performed between all sample runs to eliminate carryover.

### Thermal Proteome Profiling

### Sample Preparation

E. coli samples at 37° C. (+AZM and −AZM) and 70° C. (+AZM and −AZM) were used to perform two-temperature-point TPP. 10 μL of each sample was incubated with 100 μL of 6 M urea and 5 μL of 200 mM DTT for denaturation and reduction respectively at 37° C. for 1 h followed by incubation with 20 μL of 200 mM iodoacetamide for alkylation at room temperature in the dark for 30 min. After alkylation, proteins were incubated with 100 μL of 200 mM DTT at room temperature immediately followed by addition of 25 mM ABC to a final volume of 1 mL. Proteins were digested with trypsin (prepared in 25 mM ammonium bicarbonate) at 37° C. and pH 7 overnight with a protein to enzyme ratio of 50:1 (w/w). The digested peptides were desalted using a Strata C18-U column (55 m, 70 Å, 100 mg/mL) before vacuum concentration. The dried peptides were dissolved into 100 μL HPLC water and stored at −80° C. prior to LC-MS/MS analysis. Samples were prepared in triplicate for each condition.

### NanoLC Separation

Ten μL of protein digest was injected on a C18 RPLC column (75 μm i.d., 150 mm length, 2 μm C18 resin) for peptide separation. Mobile phase A (0.1% formic acid in HPLC water) and mobile phase B (0.1% formic acid in acetonitrile) were prepared for nanoLC separation. The flow was split to achieve a final flow rate of approximately 0.8 μL/min through the RPLC column⁵⁴. The LC gradient was started with 0% mobile phase B for sample loading for 30 min followed by an increase to 40% mobile phase B for 60 min. Then the gradient was further increased to 90% mobile phase B in 3 min and was held for 5 min followed by a decrease to 0% mobile phase B in 2 min and re-equilibration of the column for 30 min. The eluted peptides were detected using an Orbitrap Exploris 240 mass spectrometer (Thermo Fisher Scientific, Bremen, Germany). A blank run with no sample injection was included between runs to eliminate carryover.

### Isothermal Titration Calorimetry

A Malvern MicroCal PEAQ-ITC instrument (Malvern Instruments Limited, UK) was used for ITC experiments. The experiments were performed at a constant temperature of 25° C. The reference power was set at 10 μcal/s. The stir speed was kept constant at 720 rpm. A stock solution of Call was prepared to a concentration of 50 μM in 1×PBS. Stock AZM solution was also prepared in 1×PBS to a concentration of 1 mM. Stock Call samples were aliquoted for heating at 37° C. and 60° C. for 5 minutes using a PTC-200 Thermal Cycler. Samples were then allowed to return to room temperature for 3 minutes before being cooled on ice to 4° C. All tubes were centrifuged at 10,000 rpm for 30 minutes at 4° C. The supernatants were taken for ITC experiments. A total of 300 μL of Call was loaded in sample cell. The titration experiments consisted of a 150-s pre-titration delay followed by 18 injections of 2 μl aliquots of 1 mM AZM solutions with 150 s intervals. The same 1×PBS buffer titrated with the corresponding ligand was served as the blank. The resulting binding isotherm were fitted by using “one set of sites” algorithms in MicroCal PEAQ-ITC analysis software.

### MS Analysis

### MS Analysis for HDX-MS

An Orbitrap Exploris 240 mass spectrometer (Thermo Fisher Scientific, Bremen, Germany) coupled with a normal flow ion source was used for peptide detection. The heated capillary was set to 320° C. with a spray voltage of 3.5 kV. Sheath gas and Aux gas were set to 35 L/min and 7 L/min respectively for improving ionization. MS scans were acquired with a resolution setting of 120,000 and 350 to 1350 m/z range. For HDX labeled samples, only LC-MS analysis was performed. The AGC for MS scans was set to standard mode, and the max ion time was set to 1000 ms with 2 micro scans. MS/MS scans were acquired with a resolution setting of 30,000 and 150 to 2000 m/z range with HCD at a normalized collision energy setting of 30%. The data-dependent acquisition mode was used for MS/MS. The AGC for MS/MS was set to standard mode, and the max ion injection time was set to auto mode with 2 micro scans. The cycle time for MS and MS/MS analysis was 2 s.

### MS Analysis for Thermal Proteome Profiling

An Orbitrap Exploris 240 mass spectrometer (Thermo Fisher Scientific, Bremen, Germany) coupled with a nano ion source was used for peptide detection. The heated capillary was set to 275° C. with a spray voltage of 2 kV. The settings of MS and MS/MS scans were the same as in HDX-MS analysis.

### Data Analysis

An E. coli proteome database (E. coli K12) downloaded from www.uniprot.org (proteome ID: UP000000625) was combined with the Call sequence (Accession number: P00921) to serve as the protein database for the following data analyses.

### HDX-MS Data Analysis

The LC-MS/MS data of non-deuterated samples was searched against the protein database using MSGF+⁵⁵ to identify E. coli lysate peptides. The decoy database was automatically generated in MSGF+ software. Methionine oxidation and N-terminal acetylation were set as variable modifications for peptide identification. In-house developed software that fits peptide mass distributions to a Gaussian model and calculates the R² was used to identify deuterated peptides and calculate the deuterium uptake of each identified peptide^{2, 19}. Peptides were manually checked to ensure that the correct isotopic patterns were taken to calculate the deuterium uptake of the corresponding peptides. Deuterated peptides identified in all triplicate runs and both labeling time conditions were taken into statistical analysis. A two-tailed t-test was applied to determine the likelihood that the deuterium uptake difference between −AZM and +AZM were significantly different.⁵⁶

### TPP Data Analysis

All raw files from thermal proteome profiling were processed using MaxQuant (version 1.6.5.0) for peptide identification and quantification⁵⁷. Default MaxQuant parameters were used unless otherwise noted. Methionine oxidation and N-terminal acetylation were set as variable modifications and carbamidomethylation of cysteine residues was selected as a fixed modification. Identified peptides were filtered for a maximum FDR of 0.01. The match between run features was selected with a match window of 0.7 min and an alignment window of 20 min. The identified contaminants and reverse sequences were filtered out. Only peptides quantified in all three replicates runs with LFQ values larger than 0 in each condition were included for the following statistical analysis.

### Results

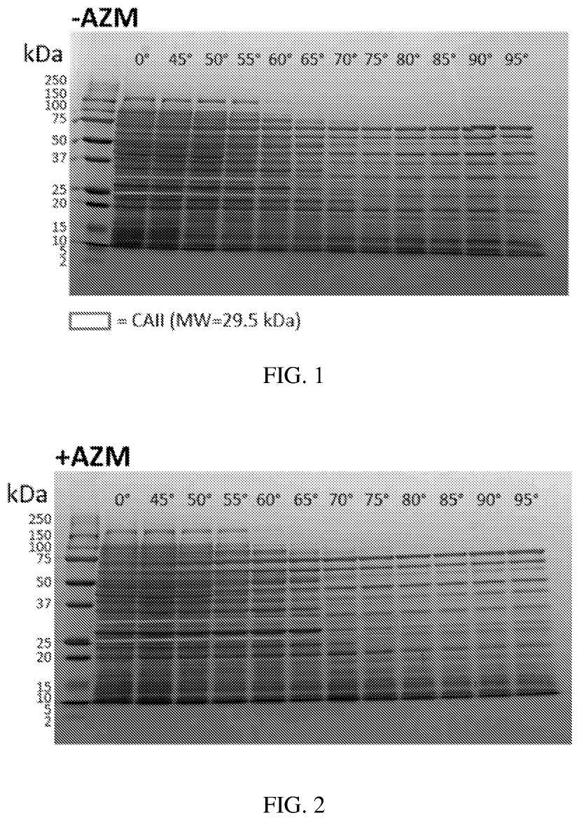

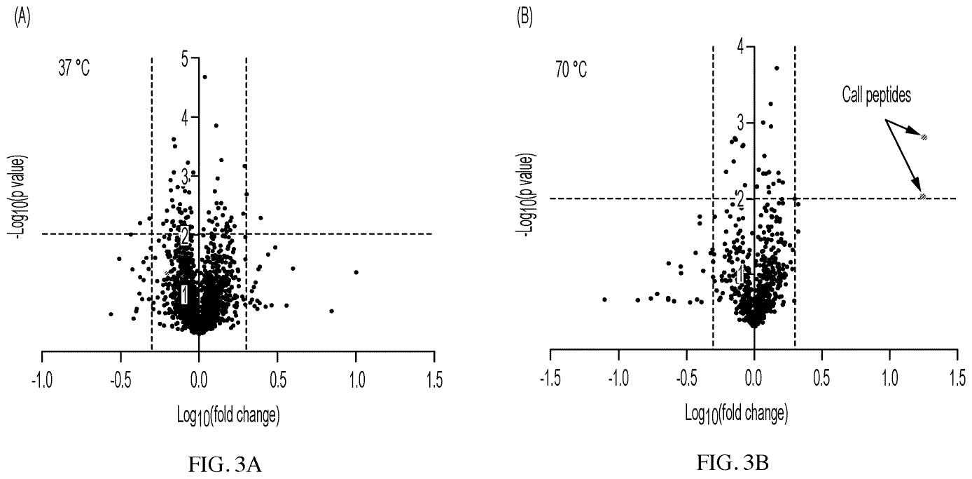

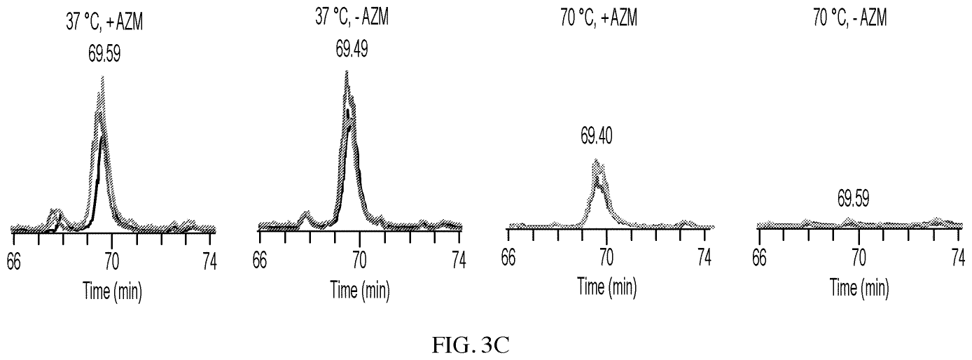

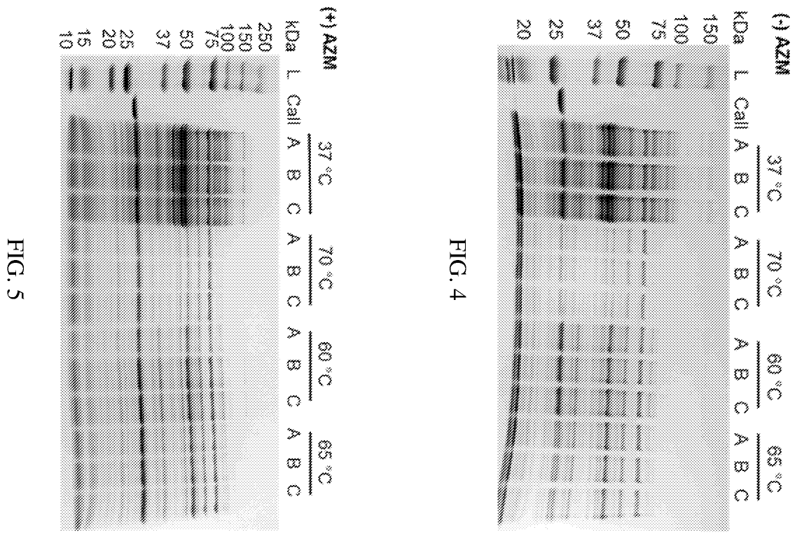

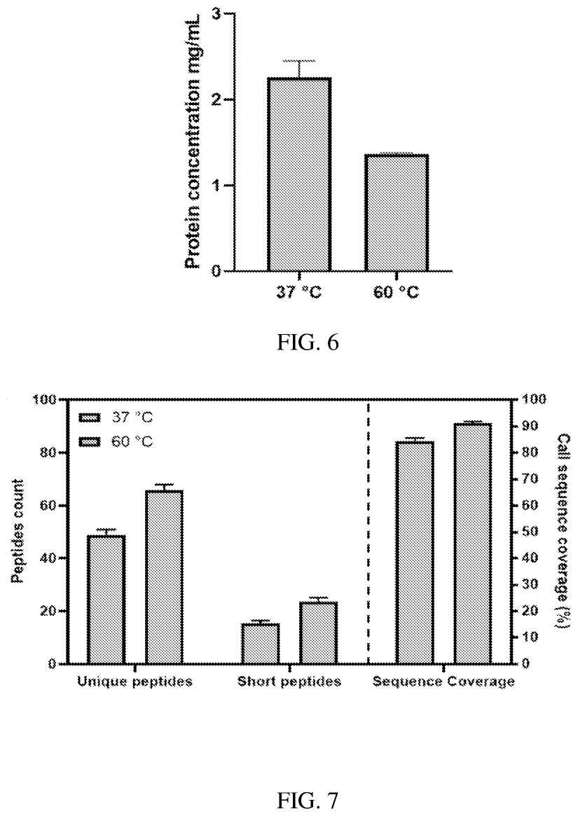

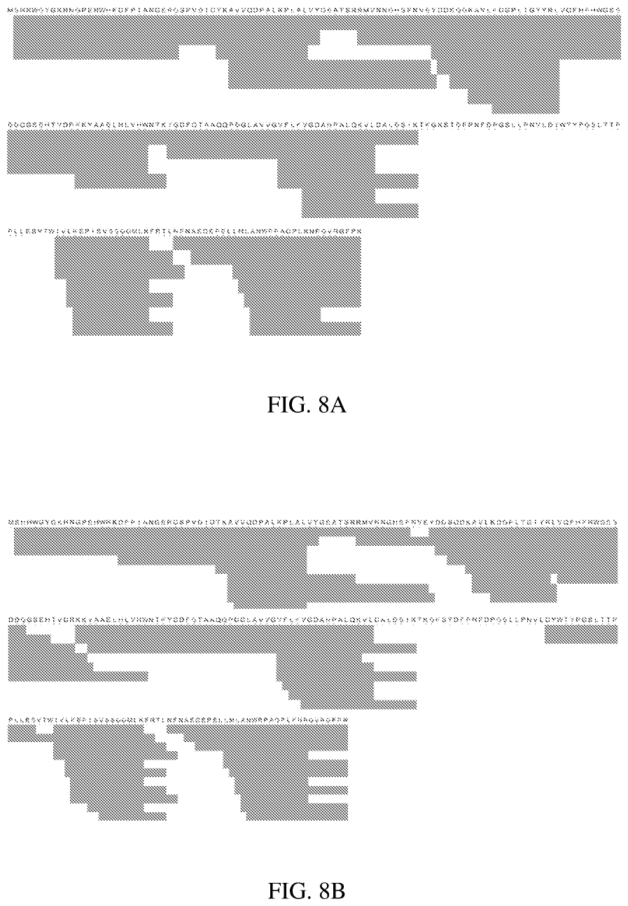

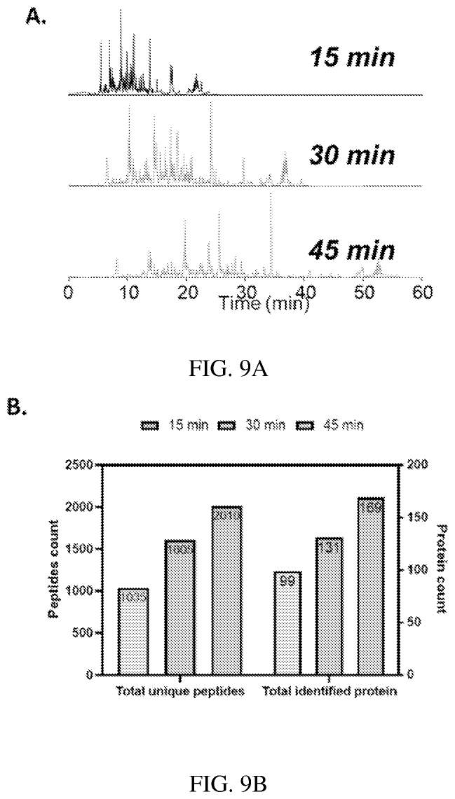

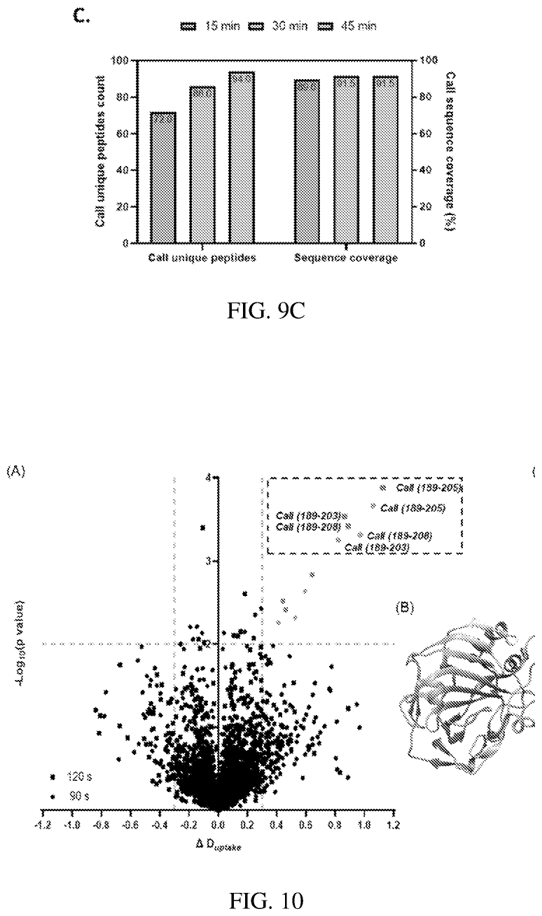

As a proof-of-concept for the proposed PTD-HDX-MS platform, E. coli cell lysate spiked with bovine Call and its ligand AZM was used as the pairing model in our study. To determine the appropriate temperature for PTD, we heated both −AZM and +AZM aliquots to temperatures from 45° C. to 95° C. in increments of 5° C. Our preliminary results suggested Call remained in solution at 60° C. and denatured at 65° C. in the −AZM set (FIG. 1) while Call remained in solution at 65° C. and denatured at 70° C. in the +AZM samples (FIG. 2). This Tₘ shift indicates that Call was stabilized upon AZM binding. Our label-free, two-temperature point bottom-up TPP results also verified that AZM targeted Call in E. coli cell lysate (FIG. 3). We then performed triplicate PTD at 60° C., 65° C., and 70° C. Subsequent SDS-PAGE analysis FIGS. 4-5 showed an identical protein distribution between triplicate PTD runs for each temperature, indicating the reproducibility of the PTD process. Compared with the protein pattern at 37° C., some protein bands were diminished or disappeared completely following PTD while Call still remained in solution at 60° C. −AZM (FIG. 5) and at 65° C. +AZM (FIG. 5). These results indicate that the protein complexity was greatly reduced by the removal of some untargeted proteins in PTD. It has also been reported that free Call still remains functional for catalytic activity after heating at 60° C. for 5 minutes³¹. Therefore, −AZM and +AZM E. coli samples exposed to PTD at 60° C. were used for the following characterizations. BCA measurement suggested that approximately 40% of the protein was removed in 60° C.-depleted E. coli cell lysate (FIG. 6). We then further evaluated the identification of Call peptides in both E. coli cell lysate without PTD (37° C.) and E. coli cell lysate with PTD at 60° C. following typical HDX-MS workflow (FIGS. 7, 8A, 8B). 66±2 unique Call peptides were identified in 60° C.-depleted E. coli cell lysate and 35% fewer unique Call peptides (49±2) were identified in E. coli cell lysate without PTD. 91.5±0.5% sequence coverage of Call was achieved in 60° C.-depleted E. coli cell lysate, approximately 7% higher than the sequence coverage obtained in E. coli cell lysate without PTD. Moreover, 55% of the additional unique peptides less than 16 amino acids in length were identified in 60° C.-depleted E. coli cell lysate compared to those peptides identified in E. coli cell lysate without PTD. These shorter peptides are essential for increasing the resolution of the characterization of the protein-ligand binding site in native-like conditions. Overall, our results demonstrate that protein thermal depletion greatly reduces protein complexity in cell lysates, enabling more peptide identification and improved sequence coverage of the targeted protein. The improved results will be advantageous for the binding site characterization in the following HDX-MS analysis.

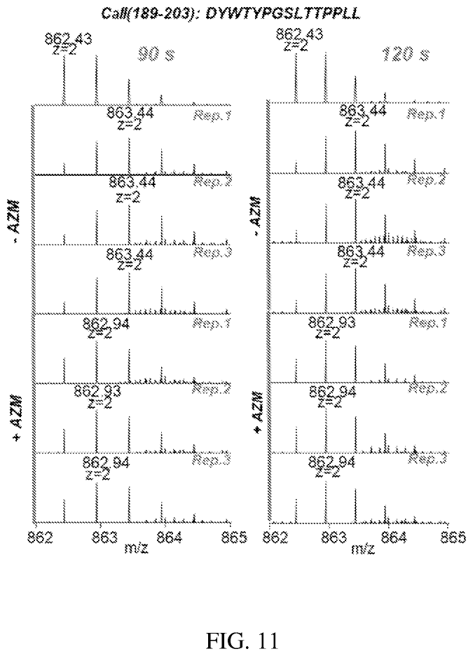

Increasing proteome coverage and peptide identification count are essential for drug target identification and binding site elucidation in HDX-MS analyses. Using E. coli digest, we previously demonstrated that subzero temperature, long gradient UPLC separation can be applied in HDX-MS analysis for improved peptide identification and proteome coverage with low deuterium back exchange¹⁹. Here, before performing HDX-MS analysis on 60° C.-depleted E. coli cell lysate, we optimized our gradient length (15 min to 45 min) for subzero temperature UPLC separation using the 60° C.-depleted E. coli cell lysate −AZM to obtain higher E. coli proteome coverage and peptide identification count. The base peak chromatographs using different gradient lengths are presented in (FIGS. 9A-9C). With the 45 min gradient, we identified 2010 peptides from 169 proteins, approximately 2 times the number of identified peptides in the 15 min gradient LC run. Increasing the LC gradient length also enabled the identification of additional Call peptides.

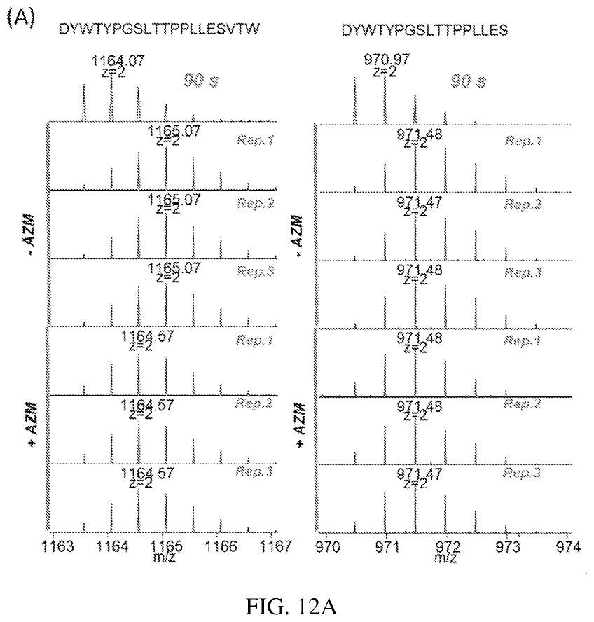

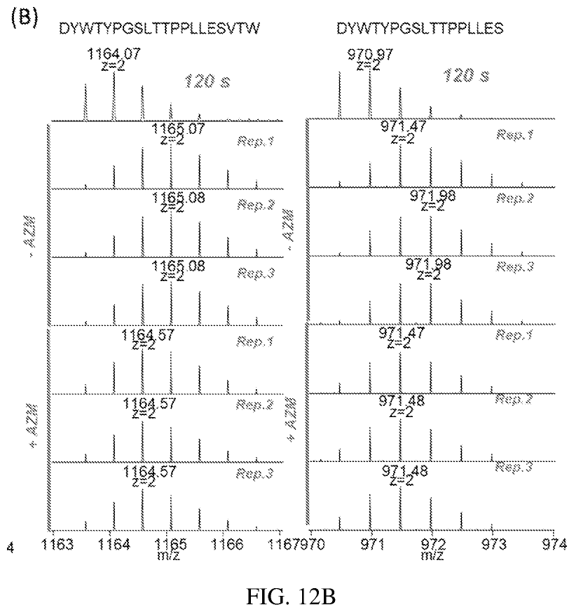

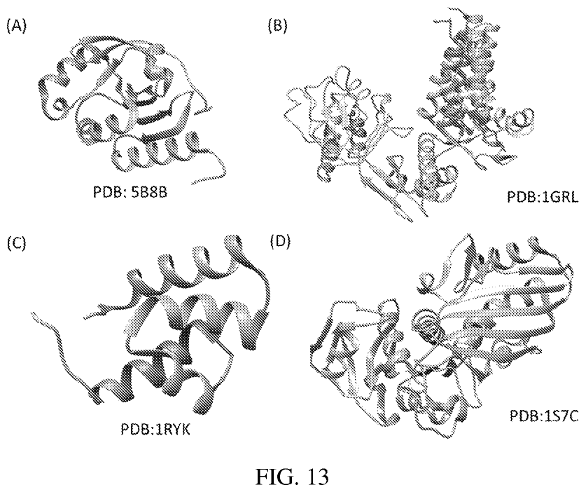

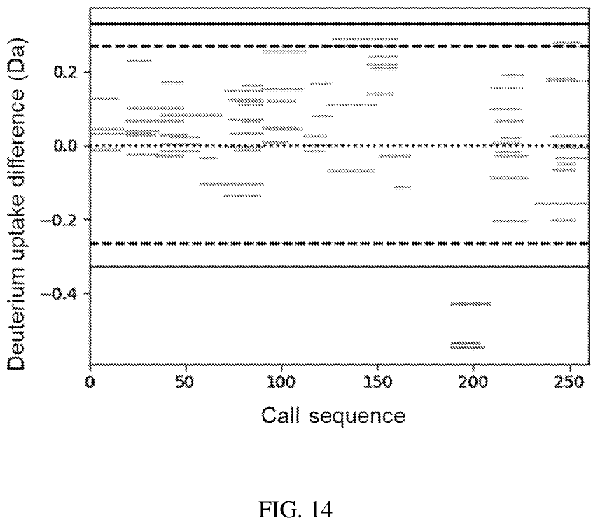

We then performed differential HDX-MS for the elucidation of both the AZM target and mechanism-of-action using 60° C.-depleted E. coli samples and a 45-min LC gradient. We characterized 1220 peptides from 134 proteins in the deuterated samples. A previous HDX-MS study on Call and troglitazone, another Call inhibitor, reported small deuterium uptake differences between free Call and Call bound with troglitazone due to the small binding interface³². Therefore, a deuterium uptake difference greater than 0.3 Da and p-value less than 0.01 were used as the cutoff in the volcano plot for statistical analysis of the differential deuterium uptake for the characterized E. coli peptides. As shown in (FIG. 10), 6 peptides exhibited significantly reduced deuterium uptake after AZM treatment. These 6 peptides were all identified from Call and were involved in the region 189-203. It was previously reported that the amino acids Leu198, Thr199, and Thr200 are involved in the hydrophobic pocket (Leu198) and hydrophilic face (Thr199 and Thr200) of the binding cavity of Call, regions which are reported to be vital for Call ligand binding³³. Examples of MS spectra of the peptides involved in the regions 189-203, 189-205, and 189-208 are shown in FIGS. 11-12B.

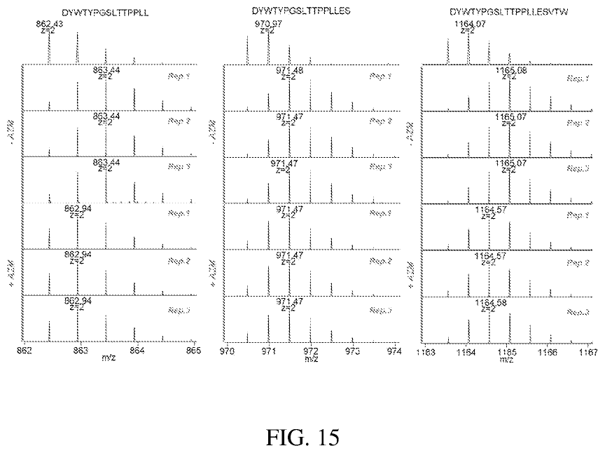

Overall, we obtained 91.54% sequence coverage of Call in the E. coli sample (FIG. 10). A few examples of HDX-MS analysis for other E. coli proteins are shown in FIG. 14. The Tₘ of alkyl hydroperoxide reductase C was reported to be 63.1° C. and the Tₘ of 60 kDa chaperonin was reported to be 68.3° C.³⁰. In 60° C.-PTD sample, an 88.24% sequence coverage and a 72.10% sequence coverage were obtained for alkyl hydroperoxide reductase C and 60 kDa chaperonin, respectively. However, no significant differences in deuterium uptake for their characterized peptides following AZM treatment were detected (FIG. 10). These results indicate that Call is the target of AZM. Particularly, the present results demonstrate that AZM targets the region 189-203 of Call to inhibit catalysis. These findings are consistent with an X-ray crystallography study of the CaII-AZM interaction which reported that one hydrogen bond is formed between AZM and Thr199 on CAII³⁴. These results show that the PTD-HDX-MS platform disclosed herein is highly sensitive and can distinguish the minor deuterium uptake differences resulting from the small binding interface between a protein and its ligand. Moreover, this is the first demonstration of the interaction between Call and AZM in a complex biological environment by the analysis of thousands of peptides from hundreds of proteins. These findings demonstrate the great utility of the application of the PTD-HDX-MS platform for the identification of unknown protein targets and the characterization of the binding site between the unknown target and ligand in complex biological samples.

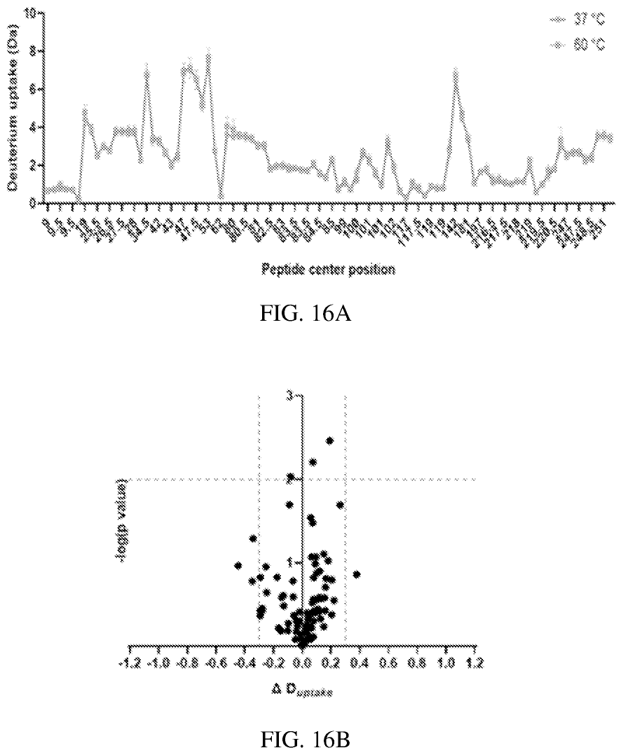

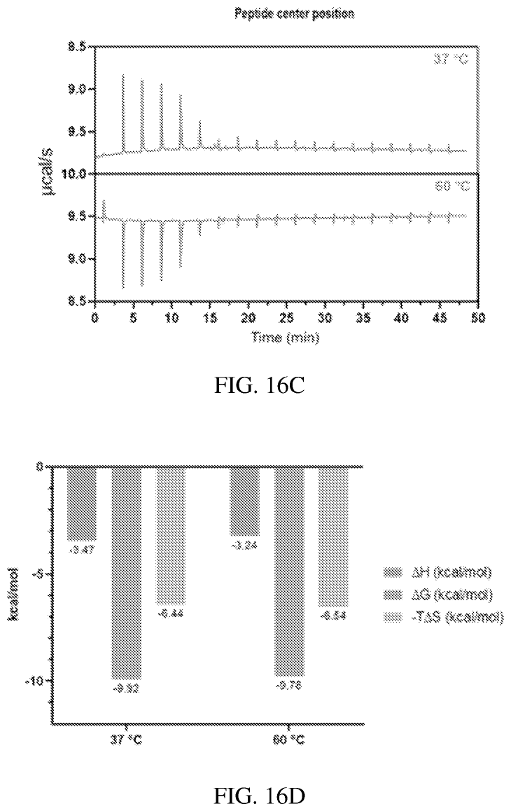

Next, to further confirm the binding site between Call and AZM, we performed HDX-MS analysis using pure Call and AZM. As shown in the Woods' plot in FIG. 14. A 91.5% sequence coverage was achieved from the pure Call sample. Three peptides covering the region 189-205 showed significantly less deuterium uptake upon AZM binding. The MS spectra of the three peptides having significantly less deuterium uptake are presented in FIG. 15. The HDX-MS results in the pure Call samples are highly consistent with the PTD-HDX-MS results from the complex E. coli samples. We then evaluated the conformational differences between Call heated to 37° C. and 60° C. to assess the conformation of Call involved in our PTD-HDX-MS analysis. As shown in FIGS. 16A-D, no significant difference in deuterium uptake was detected between Call heated at 37° C. and Call heated at 60° C. In addition, AZM binding resulted in similar ITC profiles for Call regardless of treatment temperature. These results indicate that the conformation of Call after heating to 60° C. and cooling was the same as its native form.

Generally, two steps are involved in protein thermal denaturation: the first step is reversible conformational change, and the second step is global unfolding³⁵⁻³⁷. Once the denaturation conditions are removed, proteins partially unfolded in the first step can intrinsically recover and refold to their native states given sufficient time as demonstrated by Anfinsen's dogma that the native conditions typically represent global thermodynamic minimum³⁶⁻³⁸⁻³⁹. Many studies on diverse proteins indicate that proteins refolded to their native structures after moderate heating^{37, 40-43} Upon exposure to temperatures≥Tₘ, proteins will denature and aggregate as a result of the association of exposed hydrophobic regions. A potential limitation of PTD is the possibility that some proteins may become trapped in metastable states in which conformation is misfolded to attain a local free energy minimum during refolding^{36, 44}. While this misfolded conformation may not affect differential HDX-MS analysis results when protein and protein-ligand complex are both in the same metastable states, implementation of appropriate heating temperature can help minimize local conformational changes to reduce the possibility for misfolded local conformation. Lower protein concentration in PTD can also increase the probability of proteins refolding to their native conformations prior to HDX-MS analysis³⁶. Overall, our results confirm that our PTD-HDX-MS platform coupled with subzero-temperature long gradient UPLC separation can accomplish high-throughput HDX-MS analyses for both unbiased identification of ligand targets as well as elucidation of the mechanism of protein-ligand interactions in cell lysate.

Here, we have integrated protein thermal depletion with subzero-temperature long gradient UPLC-HDX-MS (PTD-HDX-MS) to form a novel platform and demonstrated the implementation of the PTD-HDX-MS platform for the unbiased characterization of the interaction of Call and AZM in E. coli cell lysate. The present results demonstrate that heating complex biological samples such as cell lysates at a predetermined temperature can help deplete untargeted proteins, thereby greatly reducing sample complexity while the targeted protein remains in solution. Coupled with subzero temperature long gradient UPLC to boost LC separation power, the PTD-HDX-MS platform enables the high-throughput analysis of thousands of peptides from hundreds of proteins in single HDX-MS analysis. These results highlight the great potential and utility of the PTD-HDX-MS platform for the identification of ligand targets and characterization of protein-ligand interactions in highly complex biological samples.

### Determination of the Heating Temperature for PTD

In certain embodiments of the methods and systems of the present disclosure, the Tₘ, of certain target proteins is known. This previously known Tₘ can thus be used as a starting point for determining the PTD temperature to be used in the presently disclosed PTD-HDX-MS system and method. When targeted proteins are unknown, determining and implementing appropriate depletion temperatures can be challenging. In this case, ligand-targeted MS detection or ligand-specific spectroscopy analysis (e.g., monitoring ligand concentrations in temperature-gradient samples) can be performed to select the optimal depletion temperature. Alternatively, TPP approaches can be implemented for protein target identification before the application of PTD-HDX-MS to cross-validate the identified targets as well as remove random targets from non-specific binding. This can be advantageous for PTD-HDX-MS over other prefractionation methods (e.g., IEC and SEC) for HDX-MS analysis.

Determination of the Heating Temperature for PTD when Target Protein is Unknown

In certain embodiments of the present disclosure, when the ligand is known but the protein targeted by the ligand is unknown, the heating temperature for the PTD step, specific for the ligand, can be determined using the following process. First, the cell lysate sample is treated with the ligand under conditions and for a duration that enables the ligand to bind to target proteins in the sample. The ligand-treated sample is then aliquoted, and each aliquot is heated to a specific temperature selected from a predetermined range, such as 45° C. to 75° C., at a predetermined interval. The intervals used may include, but are not limited to, 0.1° C., 0.5° C., 1° C., 2° C., 3° C., 4° C., and 5° C. After each aliquot is heated to the designated, predetermined, temperature, the ligand concentration in each treated aliquot is measured using standard techniques able to measure the ligand. As the quantity of ligand in the sample diminishes, the target protein is also assumed to have been removed from the sample as well. The standard techniques for measuring the ligand concentration in the treated sample, after removal of precipitated proteins, include, but are not limited to, ligand-targeted MS detection, western blot, ligand-specific florescence spectroscopy analysis⁵¹, quantitative NMR spectroscopy⁵³, and other methods suitable for detecting the particular ligand of choice. A ligand concentration curve (that would tend to be sigmoidal in nature with a decrease in ligand concentration around the Tₘ of the target protein) can be plotted to indicate the presence of the targeted protein(s). The selected heating temperature is the highest (maximum) temperature before the ligand concentration begins declining. Isothermal titration calorimetry (ITC)⁵³ can be used to directly observe the binding of a ligand in PTD-depleted samples. At the temperature at which the response to added ligand decreases, the target protein can be assumed to have been removed from the solution. In this case, the highest (maximum) temperature before the response to the ligand begins to decrease will be selected as the PTD heating temperature.

Enhancement of protein digestion in cell lysates and implementation of DIA acquisition in HDX-MS can be used to help boost peptide identification to achieve higher proteome sequence coverage for more complex samples (e.g., mammalian cell lysates). However, it is typically challenging for HDX-MS to distinguish differential uptakes due to direct ligand binding, allosteric changes, or ligand-induced protein-protein interaction. PTD-HDX-MS may thus be used with TPP methods or ABPP⁴⁸ to cross verify the identified protein targets and eliminate false positive results; complementary methods such as chemical cross-linking and mutagenesis can be employed to reveal the mechanism of action of ligands in cell lysates⁴⁹. In addition, TPCA⁵⁰ can be paired with the presently disclosed PTD-HDX-MS process to discern induced protein-protein interactions as the result of overexpressing a protein in solution from protein-ligand interactions to see if the change in deuterium uptake relates to coaggregating proteins. By benefiting from both reduced sample complexity and improved LC separation power, the disclosed PTD-HDX-MS platform will promote the routine characterization of protein-ligand interaction in cell lysates and other complex samples such as serum and plasma to facilitate direct HDX-MS analysis of clinical samples.

### Other Applications of PTD-HDX-MS

In addition to the application of PTD-HDX-MS to study protein-drug interaction in cell lysate, protein-protein interactions, protein-metabolite interactions, protein-DNA or protein-RNA interactions can also be studied. These interactions can be studied in vivo or in vitro where living cells, cell lysate, or other complex biological systems under different conditions followed by PTD-HDX-MS. Furthermore, this technique can be applied to other complex biological systems, such as human serum, to characterize antigen-antibody interaction (e.g., epitope mapping of polyclonal antibodies in vaccine-elicited serum).

### Automation

The PTD-HDX-MS system and method can be automated to create a complete platform. In a non-limiting example, to perform PTD-HDX-MS, the system may comprise a chamber that can control the temperature to heat the sample containing proteins, and centrifugation means, such as a centrifuge, for precipitating the denatured proteins from the sample. The LC separation chamber must also be temperature controlled to facilitate low temperature separation (e.g., −10° C.).

In certain embodiments the present disclosure is directed to a method of characterizing the binding of a ligand to a protein in a biological sample, comprising the steps of (1) providing the biological sample; (2) treating the biological sample with a ligand and incubating the treated biological sample to allow the ligand to bind to a target protein, thereby forming a ligand-target protein complex in the treated biological sample; (3) heating the treated biological sample to a predetermined temperature, thereby forming a heated mixture, wherein the predetermined temperature is the maximum temperature to which the ligand-target protein complex in the heated mixture can be exposed without causing denaturation of the target protein thereof; (4) reducing the temperature of the heated mixture to about room temperature or below; (5) treating the heated mixture to remove denatured proteins therefrom, forming a depleted protein mixture; (6) subjecting the depleted protein mixture to hydrogen-deuterium exchange followed by a protein digestion step to form a peptide mixture; (7) subjecting the peptide mixture to liquid chromatography forming eluted peptides; and (8) using mass spectrometry to analyze the eluted peptides. In certain embodiments, the biological sample is a cell lysate, a sample of live cells, wherein the sample of live cells is subjected to a lysing step to form a cell lysate before the heating step, or a protein mixture. The ligand may be a small molecule such as a dye, stain, or therapeutic drug, an antigen, a protein such as a peptide, oligopeptide, polypeptide, antibody, or antibody fragment, or a nucleic acid such as a DNA or RNA. The temperature of the heated mixture may be reduced, in non-limiting embodiments, to a temperature in a range of about 2° C. to about 25° C., or about 4° C. to about 20° C.

In certain embodiments the present disclosure is directed to a method of characterizing the binding of a ligand to a protein in a cell lysate, comprising the steps of (1) providing a cell lysate; (2) treating the cell lysate with a ligand and incubating the treated cell lysate to allow the ligand to bind to a target protein, thereby forming a ligand-target protein complex; (3) heating the treated cell lysate to a predetermined temperature, thereby forming a heated mixture, wherein the predetermined temperature is the maximum temperature to which the ligand-target protein complex can be exposed without causing denaturation of the target protein thereof; (4) reducing the temperature of the heated mixture to about room temperature or below; (5) treating the heated mixture to remove denatured proteins therefrom, forming a depleted protein mixture; (6) subjecting the depleted protein mixture to hydrogen-deuterium exchange followed by a protein digestion step to form a peptide mixture; (7) subjecting the peptide mixture to liquid chromatography forming eluted peptides; and (8) using mass spectrometry to analyze the eluted peptides. The ligand may be a small molecule such as a dye, stain, or therapeutic drug, an antigen, a protein such as a peptide, oligopeptide, polypeptide, antibody, or antibody fragment, or a nucleic acid such as a DNA or RNA. The temperature of the heated mixture may be reduced, in non-limiting embodiments, to a temperature in a range of about 2° C. to about 25° C., or about 4° C. to about 20° C.

In certain embodiments the present disclosure is directed to a method of characterizing the binding of a ligand to a protein in a sample of live cells, comprising the steps of (1) providing a sample of live cells; (2) treating the sample of live cells with a ligand and incubating the treated sample of live cells to allow the ligand to bind to a target protein, thereby forming a ligand-target protein complex; (3) subjecting the treated sample of live cells to a lysing step to from a cell lysate; (4) heating the cell lysate to a predetermined temperature, thereby forming a heated mixture, wherein the predetermined temperature is the maximum temperature to which the ligand-target protein complex in the heated mixture can be exposed without causing denaturation of the target protein thereof; (5) reducing the temperature of the heated mixture to about room temperature or below; (6) treating the heated mixture to remove denatured proteins therefrom, forming a depleted protein mixture; (7) subjecting the depleted protein mixture to hydrogen-deuterium exchange followed by a protein digestion step to form a peptide mixture; (8) subjecting the peptide mixture to liquid chromatography forming eluted peptides; and (9) using mass spectrometry to analyze the eluted peptides. The ligand may be a small molecule such as a dye, stain, or therapeutic drug, an antigen, a protein such as a peptide, oligopeptide, polypeptide, antibody, or antibody fragment, or a nucleic acid such as a DNA or RNA. The temperature of the heated mixture may be reduced, in non-limiting embodiments, to a temperature in a range of about 2° C. to about 25° C., or about 4° C. to about 20° C.

In any of the above embodiments, the predetermined temperature may be identified specifically for the ligand in a determination process performed prior to the heating step.

In certain embodiments the present disclosure is directed to an apparatus for performing the method of any of the above embodiments.

While the present disclosure has been described in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the presently disclosed methods and compositions. Changes may be made in the structures of the various components described herein, or the methods described herein without departing from the spirit and scope of the present disclosure.

### REFERENCES

1. Masson, G. R.; Burke, J. E.; Ahn, N. G.; Anand, G. S.; Borchers, C.; Brier, S.; Bou-Assaf, G. M.; Engen, J. R.; Englander, S. W.; Faber, J.; Garlish, R.; Griffin, P. R.; Gross, M. L.; Guttman, M.; Hamuro, Y.; Heck, A. J. R.; Houde, D.; Iacob, R. E.; Jorgensen, T. J. D.; Kaltashov, I. A.; Klinman, J. P.; Konermann, L.; Man, P.; Mayne, L.; Pascal, B. D.; Reichmann, D.; Skehel, M.; Snijder, J.; Strutzenberg, T. S.; Underbakke, E. S.; Wagner, C.; Wales, T. E.; Walters, B. T.; Weis, D. D.; Wilson, D. J.; Wintrode, P. L.; Zhang, Z.; Zheng, J.; Schriemer, D. C.; Rand, K. D., Recommendations for performing, interpreting and reporting hydrogen deuterium exchange mass spectrometry (HDX-MS) experiments. Nat Methods 2019, 16 (7), 595-602.
2. Fang, M.; Wang, Z.; Norris, K.; James, J. A.; Wu, S.; Smith, K., Hydrogen-Deuterium Exchange Mass Spectrometry Reveals a Novel Binding Region of a Neutralizing Fully Human Monoclonal Antibody to Anthrax Protective Antigen. Toxins (Basel) 2022, 14 (2).
3. Konermann, L.; Pan, J.; Liu, Y. H., Hydrogen exchange mass spectrometry for studying protein structure and dynamics. Chem Soc Rev 2011, 40 (3), 1224-34.
4. James, E. I.; Murphree, T. A.; Vorauer, C.; Engen, J. R.; Guttman, M., Advances in Hydrogen/Deuterium Exchange Mass Spectrometry and the Pursuit of Challenging Biological Systems. Chem Rev 2022, 122 (8), 7562-7623.
5. Liu, X. R.; Zhang, M. M.; Gross, M. L., Mass Spectrometry-Based Protein Footprinting for Higher-Order Structure Analysis: Fundamentals and Applications. Chem Rev 2020, 120 (10), 4355-4454.
6. Guo, C.; Steinberg, L. K.; Henderson, J. P.; Gross, M. L., Organic Solvents for Enhanced Proteolysis of Stable Proteins for Hydrogen-Deuterium Exchange Mass Spectrometry. Anal Chem 2020, 92 (17), 11553-11557.
7. Zhang, H. M.; McLoughlin, S. M.; Frausto, S. D.; Tang, H.; Emmett, M. R.; Marshall, A. G., Simultaneous reduction and digestion of proteins with disulfide bonds for hydrogen/deuterium exchange monitored by mass spectrometry. Anal Chem 2010, 82 (4), 1450-4.
8. Yan, X.; Zhang, H.; Watson, J.; Schimerlik, M. I.; Deinzer, M. L., Hydrogen/deuterium exchange and mass spectrometric analysis of a protein containing multiple disulfide bonds: Solution structure of recombinant macrophage colony stimulating factor-beta (rhM-CSFbeta). Protein Sci 2002, 11 (9), 2113-24.
9. Mysling, S.; Salbo, R.; Ploug, M.; Jorgensen, T. J., Electrochemical reduction of disulfide-containing proteins for hydrogen/deuterium exchange monitored by mass spectrometry. Anal Chem 2014, 86 (1), 340-5.
10. Wu, Y.; Engen, J. R.; Hobbins, W. B., Ultra performance liquid chromatography (UPLC) further improves hydrogen/deuterium exchange mass spectrometry. J Am Soc Mass Spectrom 2006, 17 (2), 163-7.
11. Wales, T. E.; Fadgen, K. E.; Gerhardt, G. C.; Engen, J. R., High-speed and high-resolution UPLC separation at zero degrees Celsius. Anal Chem 2008, 80 (17), 6815-20.
12. Sheff, J. G.; Hepburn, M.; Yu, Y.; Lees-Miller, S. P.; Schriemer, D. C., Nanospray HX-MS configuration for structural interrogation of large protein systems. Analyst 2017, 142 (6), 904-910.
13. Engen, J. R.; Wales, T. E., Analytical Aspects of Hydrogen Exchange Mass Spectrometry. Annu Rev Anal Chem (Palo Alto Calif) 2015, 8, 127-48.
14. Pascal, B. D.; Willis, S.; Lauer, J. L.; Landgraf, R. R.; West, G. M.; Marciano, D.; Novick, S.; Goswami, D.; Chalmers, M. J.; Griffin, P. R., HDX workbench: software for the analysis of H/D exchange MS data. J Am Soc Mass Spectrom 2012, 23 (9), 1512-21.
15. Guttman, M.; Weis, D. D.; Engen, J. R.; Lee, K. K., Analysis of overlapped and noisy hydrogen/deuterium exchange mass spectra. J Am Soc Mass Spectrom 2013, 24 (12), 1906-12.
16. Raval, S.; Sarpe, V.; Hepburn, M.; Crowder, D. A.; Zhang, T.; Viner, R.; Schriemer, D. C., Improving Spectral Validation Rates in Hydrogen-Deuterium Exchange Data Analysis. Anal Chem 2021, 93 (9), 4246-4254.
17. Trabjerg, E.; Nazari, Z. E.; Rand, K. D., Conformational analysis of complex protein states by hydrogen/deuterium exchange mass spectrometry (HDX-MS): Challenges and emerging solutions. TrAC Trends in Analytical Chemistry 2018, 106, 125-138.
18. Wales, T. E.; Fadgen, K. E.; Eggertson, M. J.; Engen, J. R., Subzero Celsius separations in three-zone temperature controlled hydrogen deuterium exchange mass spectrometry. J Chromatogr A 2017, 1523, 275-282.
19. Fang, M.; Wang, Z.; Cupp-Sutton, K. A.; Welborn, T.; Smith, K.; Wu, S., High-throughput hydrogen deuterium exchange mass spectrometry (HDX-MS) coupled with subzero-temperature ultrahigh pressure liquid chromatography (UPLC) separation for complex sample analysis. Anal Chim Acta 2021, 1143, 65-72.
20. Walters, B. T.; Ricciuti, A.; Mayne, L.; Englander, S. W., Minimizing back exchange in the hydrogen exchange-mass spectrometry experiment. J Am Soc Mass Spectrom 2012, 23 (12), 2132-9.
21. Calvaresi, V.; Redsted, A.; Norais, N.; Rand, K. D., Hydrogen-Deuterium Exchange Mass Spectrometry with Integrated Size-Exclusion Chromatography for Analysis of Complex Protein Samples. Anal Chem 2021, 93 (33), 11406-11414.
22. Jensen, P. F.; Jorgensen, T. J.; Koefoed, K.; Nygaard, F.; Sen, J. W., Affinity capture of biotinylated proteins at acidic conditions to facilitate hydrogen/deuterium exchange mass spectrometry analysis of multimeric protein complexes. Anal Chem 2013, 85 (15), 7052-9.
23. Michalski, A.; Cox, J.; Mann, M., More than 100,000 detectable peptide species elute in single shotgun proteomics runs but the majority is inaccessible to data-dependent LC-MS/MS. J Proteome Res 2011, 10 (4), 1785-93.
24. Coales, S. J.; Tuske, S. J.; Tomasso, J. C.; Hamuro, Y., Epitope mapping by amide hydrogen/deuterium exchange coupled with immobilization of antibody, on-line proteolysis, liquid chromatography and mass spectrometry. Rapid Commun Mass Spectrom 2009, 23 (5), 639-47.
25. Baerga-Ortiz, A.; Hughes, C. A.; Mandell, J. G.; Komives, E. A., Epitope mapping of a monoclonal antibody against human thrombin by HID-exchange mass spectrometry reveals selection of a diverse sequence in a highly conserved protein. Protein Sci 2002, 11 (6), 1300-8.
26. Iacob, R. E.; Murphy, J. P., 3rd; Engen, J. R., Ion mobility adds an additional dimension to mass spectrometric analysis of solution-phase hydrogen/deuterium exchange. Rapid Commun Mass Spectrom 2008, 22 (18), 2898-904.
27. Anderson, K. W.; Hudgens, J. W., Chromatography at −30 degrees C. for Reduced Back-Exchange, Reduced Carryover, and Improved Dynamic Range for Hydrogen-Deuterium Exchange Mass Spectrometry. J Am Soc Mass Spectrom 2022, 33 (7), 1282-1292.
28. Mateus, A.; Kurzawa, N.; Becher, I.; Sridharan, S.; Helm, D.; Stein, F.; Typas, A.; Savitski, M. M., Thermal proteome profiling for interrogating protein interactions. Mol Syst Biol 2020, 16 (3), e9232.
29. Martinez Molina, D.; Jafari, R.; Ignatushchenko, M.; Seki, T.; Larsson, E. A.; Dan, C.; Sreekumar, L.; Cao, Y.; Nordlund, P., Monitoring drug target engagement in cells and tissues using the cellular thermal shift assay. Science 2013, 341 (6141), 84-7.
30. Mateus, A.; Bobonis, J.; Kurzawa, N.; Stein, F.; Helm, D.; Hevler, J.; Typas, A.; Savitski, M. M., Thermal proteome profiling in bacteria: probing protein state in vivo. Mol Syst Biol 2018, 14 (7), e8242.
31. Kanbar, B.; Ozdemir, E., Thermal stability of carbonic anhydrase immobilized within polyurethane foam. Biotechnology progress 2010, 26 (5), 1474-80.
32. Mueller, S. L.; Chrysanthopoulos, P. K.; Halili, M. A.; Hepburn, C.; Nebl, T.; Supuran, C. T.; Nocentini, A.; Peat, T. S.; Poulsen, S. A., The Glitazone Class of Drugs as Carbonic Anhydrase Inhibitors-A Spin-Off Discovery from Fragment Screening. Molecules 2021, 26 (10).
33. Krishnamurthy, V. M.; Kaufman, G. K.; Urbach, A. R.; Gitlin, I.; Gudiksen, K. L.; Weibel, D. B.; Whitesides, G. M., Carbonic anhydrase as a model for biophysical and physical-organic studies of proteins and protein-ligand binding. Chem Rev 2008, 108 (3), 946-1051.
34. Fisher, S. Z.; Aggarwal, M.; Kovalevsky, A. Y.; Silverman, D. N.; McKenna, R., Neutron diffraction of acetazolamide-bound human carbonic anhydrase II reveals atomic details of drug binding. J Am Chem Soc 2012, 134 (36), 14726-9.
35. Narhi, L. O.; Philo, J. S.; Sun, B.; Chang, B. S.; Arakawa, T., Reversibility of heat-induced denaturation of the recombinant human megakaryocyte growth and development factor. Pharm Res 1999, 16 (6), 799-807.
36. Tsong, T. Y.; Gross, C. J., Reversibility of thermally induced denaturation of cellular proteins. Ann N Y Acad Sci 1994, 720, 65-78.
37. Tang, C. H.; Choi, S. M.; Ma, C. Y., Study of thermal properties and heat-induced denaturation and aggregation of soy proteins by modulated differential scanning calorimetry. Int J Biol Macromol 2007, 40 (2), 96-104.
38. Anfinsen, C. B., Principles that govern the folding of protein chains. Science 1973, 181 (4096), 223-30.
39. Dill, K. A.; Ozkan, S. B.; Shell, M. S.; Weikl, T. R., The protein folding problem. Annu Rev Biophys 2008, 37, 289-316.
40. Zhang, L.; Zhou, R.; Zhang, J.; Zhou, P., Heat-induced denaturation and bioactivity changes of whey proteins. International Dairy Journal 2021, 123, 105175.
41. Akkerman, M.; Rauh, V. M.; Christensen, M.; Johansen, L. B.; Hammershoj, M.; Larsen, L. B., Effect of heating strategies on whey protein denaturation—Revisited by liquid chromatography quadrupole time-of-flight mass spectrometry. J Dairy Sci 2016, 99 (1), 152-66.
42. Saha, P.; Manna, C.; Chakrabarti, J.; Ghosh, M., Reversible thermal unfolding of a yfdX protein with chaperone-like activity. Sci Rep 2016, 6, 29541.
43. Lipman, E. A.; Schuler, B.; Bakajin, O.; Eaton, W. A., Single-molecule measurement of protein folding kinetics. Science 2003, 301 (5637), 1233-5.
44. Ghosh, D. K.; Ranjan, A., The metastable states of proteins. Protein Sci 2020, 29 (7), 1559-1568.
45. Jafari, R.; Almqvist, H.; Axelsson, H.; Ignatushchenko, M.; Lundback, T.; Nordlund, P.; Martinez Molina, D., The cellular thermal shift assay for evaluating drug target interactions in cells. Nat Protoc 2014, 9 (9), 2100-22.
46. Savitski, M. M.; Reinhard, F. B.; Franken, H.; Werner, T.; Savitski, M. F.; Eberhard, D.; Martinez Molina, D.; Jafari, R.; Dovega, R. B.; Klaeger, S.; Kuster, B.; Nordlund, P.; Bantscheff, M.; Drewes, G., Tracking cancer drugs in living cells by thermal profiling of the proteome. Science 2014, 346 (6205), 1255784.
47. Herneisen, A. L.; Sidik, S. M.; Markus, B. M.; Drewry, D. H.; Zuercher, W. J.; Lourido, S., Identifying the Target of an Antiparasitic Compound in Toxoplasma Using Thermal Proteome Profiling. ACS Chem Biol 2020, 15 (7), 1801-1807.
48. Wang, S.; Tian, Y.; Wang, M.; Wang, M.; Sun, G. B.; Sun, X. B., Advanced Activity-Based Protein Profiling Application Strategies for Drug Development. Front Pharmacol 2018, 9, 353.
49. Ozohanics, O.; Ambrus, A., Hydrogen-Deuterium Exchange Mass Spectrometry: A Novel Structural Biology Approach to Structure, Dynamics and Interactions of Proteins and Their Complexes. Life (Basel) 2020, 10 (11).
50. Tan, C. S. H.; Go, K. D.; Bisteau, X.; Dai, L.; Yong, C. H.; Prabhu, N.; Ozturk, M. B.; Lim, Y. T.; Sreekumar, L.; Lengqvist, J.; Tergaonkar, V.; Kaldis, P.; Sobota, R. M.; Nordlund, P., Thermal proximity coaggregation for system-wide profiling of protein complex dynamics in cells. Science 2018, 359 (6380), 1170-1177.
51. Breen, C. J.; Raverdeau, M.; Voorheis, H. P., Development of a quantitative fluorescence-based ligand-binding assay. Sci Rep 2016, 6, 25769.
52. Wishart, D. S., Quantitative metabolomics using NMR. TrAC Trends in Analytical Chemistry 2008, 27 (3), 228-237.
53. Zhou, X.; Sun, Q.; Kini, R. M.; Sivaraman, J., A universal method for fishing target proteins from mixtures of biomolecules using isothermal titration calorimetry. Protein Sci 2008, 17 (10), 1798-804.
54. Yang, W.; Wang, L.; Fang, M.; Sheth, V.; Zhang, Y.; Holden, A. M.; Donahue, N. D.; Green, D. E.; Frickenstein, A. N.; Mettenbrink, E. M.; et al. Nanoparticle Surface Engineering with Heparosan Polysaccharide Reduces Serum Protein Adsorption and Enhances Cellular Uptake. Nano Lett 2022, 22 (5), 2103-2111. DOI: 10.1021/acs.nanolett.2c00349.
55. Kim, S.; Pevzner, P. A. MS-GF+ makes progress towards a universal database search tool for proteomics. Nat Commun 2014, 5, 5277. DOI: 10.1038/ncomms6277.
56. Strutzenberg, T. S.; Garcia-Ordonez, R. D.; Novick, S. J.; Park, H.; Chang, M. R.; Doebellin, C.; He, Y.; Patouret, R.; Kamenecka, T. M.; Griffin, P. R. HDX-MS reveals structural determinants for RORgamma hyperactivation by synthetic agonists. Elife 2019, 8. DOI: 10.7554/eLife.47172.
57. Tyanova, S.; Temu, T.; Cox, J. The MaxQuant computational platform for mass spectrometry-based shotgun proteomics. Nat Protoc 2016, 11 (12), 2301-2319. DOI: 10.1038/nprot.2016.136.

What is claimed is:

1. A method of characterizing the binding of a ligand to a protein in a biological sample, comprising the steps of:
providing the biological sample;
treating the biological sample with a ligand and incubating the treated biological sample to allow the ligand to bind to a target protein, thereby forming a ligand-target protein complex in the treated biological sample;
heating the treated biological sample to a predetermined temperature, thereby forming a heated mixture, wherein the predetermined temperature is the maximum temperature to which the ligand-target protein complex in the heated mixture can be exposed without causing denaturation of the target protein thereof;
reducing the temperature of the heated mixture to about room temperature or below;
treating the heated mixture to remove denatured proteins therefrom, forming a depleted protein mixture;
subjecting the depleted protein mixture to hydrogen-deuterium exchange followed by a protein digestion step to form a peptide mixture;
subjecting the peptide mixture to liquid chromatography forming eluted peptides; and
using mass spectrometry to analyze the eluted peptides.

2. The method of claim 1, wherein the biological sample is a cell lysate.

3. The method of claim 1, wherein the biological sample is a sample of live cells, and wherein the sample of live cells is subjected to a lysing step to form a cell lysate before the heating step.

4. The method of claim 1, wherein the biological sample is a protein mixture.

5. The method of claim 1, wherein the ligand is selected from the group consisting of a small molecule, an antigen, a protein, and a nucleic acid.

6. The method of claim 5, wherein the small molecule is selected from the group consisting of a dye, a stain, and a therapeutic drug.

7. The method of claim 5, wherein the protein is selected from the group consisting of a peptide, an oligopeptide, a polypeptide, an antibody, and an antibody fragment.

8. The method of claim 5, wherein the nucleic acid is selected from the group consisting of DNA and RNA.

9. The method of claim 1, wherein the temperature of the heated mixture is reduced to a temperature in a range of about 2° C. to about 25° C.

10. The method of claim 1, wherein the predetermined temperature is identified specifically for the ligand in a determination process performed prior to the heating step.

11. The method of claim 1, comprising identifying an amino acid sequence to which the ligand binds.

12. A method of characterizing the binding of a ligand to a protein in a sample of live cells, comprising the steps of:
providing a sample of live cells;
treating the sample of live cells with a ligand and incubating the treated sample of live cells to allow the ligand to bind to a target protein, thereby forming a ligand-target protein complex;
subjecting the treated sample of live cells to a lysing step to from a cell lysate;
heating the cell lysate to a predetermined temperature, thereby forming a heated mixture, wherein the predetermined temperature is the maximum temperature to which the ligand-target protein complex in the heated mixture can be exposed without causing denaturation of the target protein thereof;
reducing the temperature of the heated mixture to about room temperature or below;
treating the heated mixture to remove denatured proteins therefrom, forming a depleted protein mixture;
subjecting the depleted protein mixture to hydrogen-deuterium exchange followed by a protein digestion step to form a peptide mixture;
subjecting the peptide mixture to liquid chromatography forming eluted peptides; and
using mass spectrometry to analyze the eluted peptides.

13. The method of claim 12, wherein the ligand is selected from the group consisting of a small molecule, an antigen, a protein, and a nucleic acid.

14. The method of claim 13, wherein the small molecule is selected from the group consisting of a dye, a stain, and a therapeutic drug.

15. The method of claim 13, wherein the protein is selected from the group consisting of a peptide, an oligopeptide, a polypeptide, an antibody, and an antibody fragment.

16. The method of claim 13, wherein the nucleic acid is selected from the group consisting of DNA and RNA.

17. The method of claim 12, wherein the temperature of the heated mixture is reduced to a temperature in a range of about 2° C. to about 25° C.

18. The method of claim 12, wherein the predetermined temperature is identified specifically for the ligand in a determination process performed prior to the heating step.

19. The method of claim 12, comprising identifying an amino acid sequence to which the ligand binds.
